(19) United States
(12) Patent Application Publication
(10) Pub. No.: US 2013/0133400 A1
(43) Pub. Date: May 30, 2013

(54) DEVICE FOR MEASURING AT LEAST ONE EXHAUST GAS COMPONENT IN AN EXHAUST GAS DUCT OF A COMBUSTION PROCESS

(71) Applicant: Robert Bosch GmbH, Stuttgart (DE)

(72) Inventors: Hans-Martin Jaeger, Ludwigsburg (DE); Dieter Schuler, Stuttgart (DE)

(73) Assignee: ROBERT BOSCH GMBH, Stuttgart (DE)

(21) Appl. No.: 13/682,340

(22) Filed: Nov. 20, 2012

(30) Foreign Application Priority Data
Nov. 24, 2011 (DE) 10 2011 087 000.8

(51) Int. Cl. G01N 33/00

(52) U.S. Cl. CPC G01N 33/0009
USPC 73/23.31

(57) ABSTRACT

A device (10), which contains at least one exhaust gas probe (16), is proposed for measuring at least one exhaust gas component in an exhaust gas duct (14) of a combustion process. The device is characterized in that the exhaust gas probe (16) comprises at least one air connection (20), at least one air duct (60) leading past a sensing element (64) of said exhaust gas probe (16) and connected to the air connection (20) as well as at least one air outlet opening (22).

### BACKGROUND OF THE INVENTION

The invention relates to a device, which contains at least one exhaust gas probe, for measuring at least one exhaust gas component in an exhaust gas duct of a combustion process.

The German patent publication DE 198 10 483 A1 discloses a method for determining the signal offset of HC and or NOx probes as the basis for a calibration of the probe signal. In an operating state of an internal combustion engine, in which no HC or NOx emissions occur, the signal provided by the probe is classified as an offset signal and is used to calibrate the probe signal. A state in which no HC or NOx emissions occur results, for example, during an overrun fuel cutoff, in which the supply of fuel is suppressed.

The first publication of the German patent application DE 10 2009 000 298 A1 discloses a method for calibrating a probe signal provided by a lambda probe disposed in the exhaust gas duct of an internal combustion engine. The calibration takes place during a phase of the overrun fuel cutoff of the internal combustion engine, in which said combustion engine works without the supply of fuel during the overrun mode. Provision is thereby made as a special measure for a butterfly control valve in the intake area of said combustion engine to be opened and the calibration to be performed only after opening the butterfly control valve. The method described can only be used if overrun fuel cutoff phases can be counted on to occur during the operation of said combustion engine.

The first publication of the German patent application DE 10 2005 056 152 A1 likewise discloses a method for calibrating the lambda signal supplied by a wideband lambda sensor disposed in the exhaust gas duct of an internal combustion engine. In the method, the measured lambda actual value is corrected by a correction value, which is ascertained during a predetermined operating state of the internal combustion engine, in which no fuel metering occurs and the engine rotational speed of said combustion engine lies above a threshold value. The correction value is furthermore ascertained as a function of the temperature of the wideband lambda sensor during the predetermined operating state. This method can also only be used if overrun fuel cutoff phases can be counted on to occur during the operation of said combustion engine.

The first publication of the German patent application DE 198 10 973 A1 discloses a method for calibrating the signal of a lambda probe, in which the lambda probe is periodically bathed in an alternating manner with exhaust gas of a combustion process and with fresh air. The procedural approach described is suitable for calibrating the probe signal during a continuous operation of the combustion process. The fresh air is introduced into the exhaust gas area by opening a butterfly valve disposed in the exhaust gas duct. A fireplace of a building heating system or a fuel combustion taking place in an internal combustion engine is provided as the combustion process.

The first publication of the German patent application DE 10 2008 046 121 A1 discloses a method for calibrating the signal of a lambda probe or NOx probe disposed in an exhaust gas duct of an internal combustion engine. The procedural approach described is suited for calibrating the probe signal during a continuous operation of the combustion process in the internal combustion engine. The calibration is carried out when bathing air flows by the probe. In order to do this, the lambda probe is removed from the exhaust gas duct. As an alternative, ambient air is blown onto the probe by means of an air duct, which is directed into the exhaust gas duct and surrounds the part of the probe projecting into said exhaust gas duct.

The first publication of the German patent application DE 10 2010 027 984 A1 (not prepublished) discloses a method for calibrating the signal of a lambda probe disposed in an exhaust gas duct of an internal combustion engine. The method provides for ambient air to be supplied to the exhaust gas duct upstream of the probe by means of a device for supplying additional air during an operating state of the internal combustion engine in which no combustion of fuel takes place. The device for supplying additional air can be implemented within the scope of a combustion secondary air delivery system. It is again assumed here that overrun fuel cutoff phases of said combustion engine occur at least occasionally, wherein ambient air is however additionally passed by the probe during such an overrun fuel cutoff phase.

The German patent publication DE 10 2010 027 983 A1 (not prepublished) discloses a further method for calibrating the signal of a lambda probe disposed in an exhaust gas duct of an internal combustion engine, in which the calibration is carried out during the startup process of the internal combustion engine that is cranked by an electric starter motor. The fuel injection is suppressed during the calibration.

In the technical manual “Ottomotor-Management/Bosch” (“Gasoline Engine Management: Bosch”), published by Vieweg, 1. edition, 1998, pages 22-23, a wideband lambda sensor is described, which has a sensor chamber that is connected to a gas compartment via a diffusion barrier. An inner pump electrode is disposed in the sensor chamber, said inner pump electrode forming a pump cell with an outer pump electrode and an electrolyte which lies between the two pump electrodes and conducts oxygen ions. Oxygen ions can be pumped through the electrolyte out of the sensor chamber or into the sensor chamber with the pump cell. Besides the pump cell, a measuring cell is present, which lies between the inner pump electrode and a reference gas electrode, wherein an electrolyte that conducts oxygen ions is likewise disposed between the inner pump electrode and the reference gas electrode. The measuring cell corresponds to a Nernst cell, whereat the potential difference that forms in thermodynamic equilibrium between the inner pump electrode and the reference electrode is proportional to the logarithm of the ratio of the partial pressure of the gas being analyzed in the sensor chamber to the partial pressure of the gas being analyzed in the air reference. The objective of a measurement of the exhaust gas lambda is to influence the oxygen partial pressure in the sensor chamber such that the Nernst potential remains constant at a predetermined value, which preferably corresponds to lambda=1. For this purpose, a circuit arrangement changes an electric pump current with which the outer pump electrode is impinged. The polarity and the amount of pump current depends on whether and the amount by which the predetermined Nernst potential is exceeded or undershot. The resulting pump current is a measurement for the exhaust gas lambda.

The correlation between the pump current and the measurement for the exhaust gas lambda can be ascertained within the scope of a calibration.

### SUMMARY OF THE INVENTION

The aim underlying the invention is to specify a device comprising an exhaust gas probe, in particular a lambda, NOx and/or HC probe for measuring at least one exhaust gas component contained in an exhaust gas of a combustion process.

The device according to the invention for measuring at least one exhaust gas component in an exhaust gas duct of a combustion process proceeds on the assumption that at least one exhaust gas probe is provided. The inventive device is thereby characterized in that the exhaust gas probe comprises at least one air connection, at least one air duct leading past a sensing element of the exhaust gas probe and connected to the air connection as well as at least one air outlet opening connected to the at least one air duct.

The inventive device allows the sensing element of the exhaust gas probe to be impinged with ambient air in the installed state of said exhaust gas probe even during normal operation of said probe. Additional separate air ducts required to date are now eliminated.

A bathing with ambient air can be used to calibrate the exhaust gas signal provided by the exhaust gas probe if the sensing element of the exhaust gas probe provides an exhaust gas signal that is dependent on the concentration of air, for example on the oxygen content of the air. The exhaust gas signal of an exhaust gas probe embodied as a wideband lambda sensor can detect a lambda range from a rich air/fuel ratio to a very lean air/fuel ratio. In so doing, the excess air can come up to the oxygen concentration of the ambient air which lies at approximately 21%.

The calibration of such an exhaust gas probe can result from bathing the sensing element with ambient air, wherein a value, which corresponds to an oxygen content of 21%, is associated with the exhaust gas signal. A comparable calibration is possible if the exhaust gas probe is implemented as a NOx sensor or HC sensor.

The calibration that is possible with the device according to the invention, in particular during the operation of the exhaust gas probe when said probe is installed in the exhaust gas duct of the combustion process, proves to be especially advantageous when said probe is used in combustion processes which continuously occur over a comparatively long period of time.

Such operating situations occur especially in internal combustion engines, which are operated in a steady-state operating state or in a quasi-steady-state operating state. Internal combustion engines, for example, in agricultural vehicles and in construction site vehicles are usually operated continuously over several operating hours without an overrun phase occurring, in which the internal combustion engine is in fact running but the supply of fuel is cut off. Further examples are vehicles with an automatic transmission or, for example, hybrid applications. Operating situations, in which an exhaust gas flow occurs in the exhaust gas duct, which primarily consists of ambient air and could be used for calibrating the exhaust gas probe, do not occur here in each case.

Provision is made in an advantageous modification for an air supply device, which is specially prepared for connection to the at least one air connection of the exhaust gas probe. The air supply device particularly provides for the delivery of ambient air when the exhaust gas probe is installed in the exhaust gas duct of the combustion process.

One embodiment provides that the air supply device contains a valve to permit or block the supply of air. On account of this measure, a calibration of the exhaust gas probe can, for example, be initiated in certain situations, for example as a function of the operating state of the combustion process.

A basic embodiment of the air supply device provides that the air supply device contains a compressor. If it is assumed that an exhaust gas pressure which is higher than the ambient air pressure prevails in the exhaust gas duct of the combustion process, the compressor can bring about an increase in the pressure level of the ambient air; and therefore an air flow from the air supply device can occur through the exhaust gas probe into the exhaust gas duct independently of the prevailing exhaust gas pressure.

Provided that an exhaust gas turbocharger is present, the compressor can be implemented as part of the exhaust gas turbocharger. If applicable, provision is made for a discrete compressor.

Another advantageous embodiment provides that the air supply device contains an air heater. The increase in the temperature of the ambient air flowing through the exhaust gas probe can be advantageous if the sensing element of said exhaust gas probe is heated during operation to a comparatively high operating temperature in order to keep the temperature differences resulting from impinging the sensor element with ambient air and the consequent thermal stress as small as possible.

The air heater can, for example, be implemented as an electrically operated heating element. The air heater is preferably implemented by at least a part of the air supply device being disposed in the exhaust gas duct. This enables the heat energy at least in part to be obtained from the exhaust gas energy.

Provision is made in another embodiment for the at least one air outlet opening to be disposed in a region of the exhaust gas probe protruding into the exhaust gas duct. On account of this measure, an opening which is already present in this region for bathing the sensing element of the exhaust gas probe with exhaust gas can additionally be used to discharge the ambient air.

A further embodiment provides a pressure determination device, which determines a pressure difference which can occur between the exhaust gas duct and the air supply device. The compressor, which where applicable is present in the air supply device, can be actuated with a measurement provided by the pressure determination device.

A further modification provides a specially equipped control unit for operating the device according to the invention. In so doing, said control unit can relate to a discrete control unit or to a control unit which is also provided for the open-loop and closed-loop control of the combustion process.

The control unit can, for example, be provided to process and evaluate the signals supplied by the pressure determination device and/or the exhaust gas signal supplied by the exhaust gas probe. In addition, the control unit can provide control signals to the compressor and/or the air heater and/or the valve in the air supply device.

Further advantageous embodiments and modifications ensue from the following description.

### BRIEF DESCRIPTION OF THE DRAWINGS

FIG. 1 shows an inventive device according to a first exemplary embodiment,

FIG. 2 shows a longitudinal section through an exhaust gas probe of the device according to the invention and

FIG. 3 shows a further exemplary embodiment of the device according to the invention.

### DETAILED DESCRIPTION

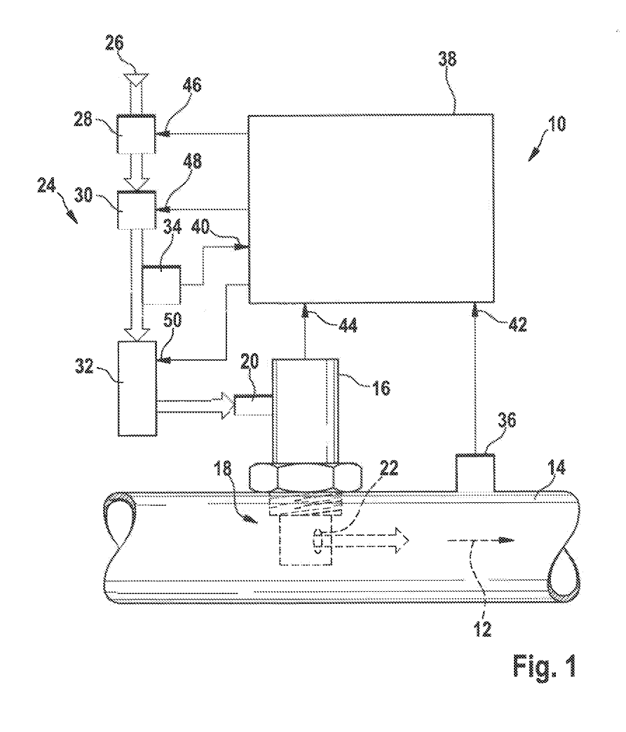

FIG. 1 shows a device 10 according to the invention for measuring at least one exhaust gas component of an exhaust gas flow 12 in an exhaust gas duct 14 of a combustion process.

The device 10 contains an exhaust gas probe 16, which is mounted at the exhaust gas duct 14 and the forward region 18 of which protrudes into said exhaust gas duct 14.

The exhaust gas probe 16 has an air connection 20 as well as an air outlet opening 22. An air supply device 24 is connected to the air connection 20, the former directing air up to said air connection 20.

The air supply device 24 contains a compressor 28, a valve 30 as well as an air heater 32. In addition, a first pressure sensor 34, which is part of a pressure determination device, is provided at the air supply device 24. The pressure determination device furthermore contains a second pressure sensor 36, which is disposed at the exhaust gas duct 14.

The first pressure sensor 34 provides a first pressure signal 40 to a control unit 38, and the second pressure sensor 36 a second pressure signal 42 to the control unit 38. In addition, the exhaust gas probe 16 provides an exhaust gas signal 44 to said control unit 38.

The control unit 38 supplies a compressor control signal 46 to the compressor 28, a valve control signal 48 to the valve 30 and an air heater control signal 50 to the air heater 32.

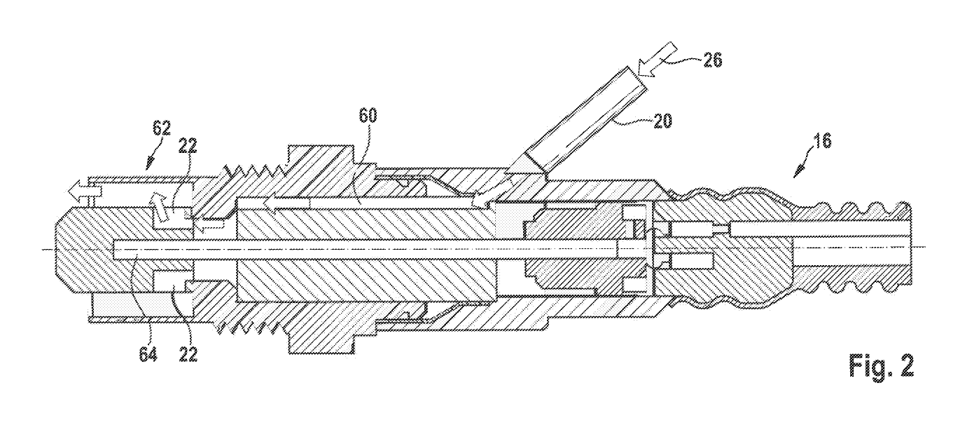

FIG. 2 shows a longitudinal section through the exhaust gas probe 16, which besides the air connection 20 comprises an air duct 60 for directing the ambient air 26, said air duct directing the ambient air 26 to the air outlet opening 22, wherein two air outlet openings 22 are shown in FIG. 2. In the exemplary embodiment of the exhaust gas probe 16 shown in FIG. 2, the air outlet opening 22 is surrounded by a protective pipe 62, which is not shown in FIG. 1. The air duct 60 runs in the exhaust gas probe 16 such that a sensing element 64 of the exhaust gas probe 16 can be bathed by the ambient air 26.

The inventive device 10 shown in FIG. 1 and pursuant to a first exemplary embodiment works in the following manner:

The inventive device 10 contains the exhaust gas probe 16 as a part, which is embodied, for example, as a lambda, NOx or HC probe. Such an exhaust gas probe 16 provides the exhaust gas signal 16 to the control unit 38, which signal reflects a measurement for the exhaust gas component being measured in the exhaust gas duct 14 of the combustion process.

In order to perform a diagnosis or a calibration of the exhaust gas probe 16, a known value of the exhaust gas component to be measured must be present in the exhaust gas flow 12. If the exhaust gas probe 16 is disposed in the exhaust gas duct 14 of a combustion process, which continues over many operating hours without interruption, the diagnosis and/or calibration of the exhaust gas probe cannot take place during this phase. Even in these operating states of the combustion process, the device 10 according to the invention succeeds in creating a diagnostic and/or calibration possibility for the exhaust gas probe 16 as a result of the sensing element 64 of the exhaust gas probe 16 being bathed in ambient air 26 without complicated additional measures having to be taken.

For this purpose, the exhaust gas probe 16 comprises the air connection 20, the air duct 60 as well as the at least one air outlet opening 22. The sensing element 64 of the exhaust gas probe 16 can therefore be exposed to ambient air 26 independently of the exhaust gas components of the exhaust gas flow. Provided that the exhaust gas signal 44 is dependent on the air concentration in the exhaust gas flow 12, preferably, for example, on the oxygen concentration in the exhaust gas flow 12, the diagnosis and/or calibration of the exhaust gas signal 44 can be performed by bathing the sensing element 64 with ambient air 26.

Bathing the sensing element 64 with ambient air ensures that the exhaust gas signal 44 has a value which reflects a measurement for the ambient air 26, for example a measurement of the oxygen concentration which lies at approximately 21%.

An increase in the pressure level of the ambient air 26 at the air connection 20 of the exhaust gas probe 16 may be required as a function of the prevailing pressure in the exhaust gas duct 14 of the combustion process. The air supply device 24, which contains the compressor 28, is therefore preferably provided as an additional part of the inventive device 10. The air supply device 24 is configured such that said air supply device 24 can be connected to the air connection 20. The compressor 28 can, for example, be a part of the exhaust gas turbocharger, provided that provision is made for an exhaust gas turbocharger in the exhaust gas duct 14. As the case may be, a discrete compressor, which is preferably electrically operated or mechanically driven (V-belt, compressed air system), can be provided.

The control unit 38 provides the compressor control signal 46 as a function of the pressure difference between ambient air 26 and the pressure in the exhaust gas duct 14, which is supplied by the pressure determination device that contains the first pressure sensor 34 disposed at the air supply device 24 and the second pressure sensor 36 disposed at the exhaust gas channel 14. The control unit 38 can ascertain the prevailing pressure difference from the pressure signals 40, 42 provided by the pressure sensors 34, 36 of the pressure determination device and correspondingly actuate the compressor 28 by means of the compressor control signal 46 so that it is possible to impinge the sensing element 64 of the exhaust gas probe 16 with ambient air 26.

If it can be assumed that an excess pressure is present in the exhaust gas channel 14 when a combustion process takes place, the valve 30, which is provided if applicable in the air supply device 24 and blocks or permits the supply of ambient air 26, can also be eliminated, wherein the ambient air 26 is only then provided if the compressor 28 is working. In so doing, the requirement is that a back flow of the exhaust gas flow 12 to the ambient 26 also does not occur without the valve 30.

The air heater 32, which is actuated by the air heater control signal 50 is preferably provided to heat up the ambient air 26 in the event that the sensing element 64 has a high temperature during the operation of the exhaust gas probe 16. In the case of the exhaust gas probe 16 being implemented, for example, as a HC, lambda or NOx probe, it must be assumed that the sensing element 64 is heated up to temperatures up to 850 degrees Celsius. In order to prevent thermal shocks as a result of impinging said sensing element 64 with cold ambient air 26, the heating of the ambient air 26 using the air heater 32 may be necessary.

The air heater 32 can, for example, be implemented as an electrically operated heating element, which can be activated with the air heater control signal 50.

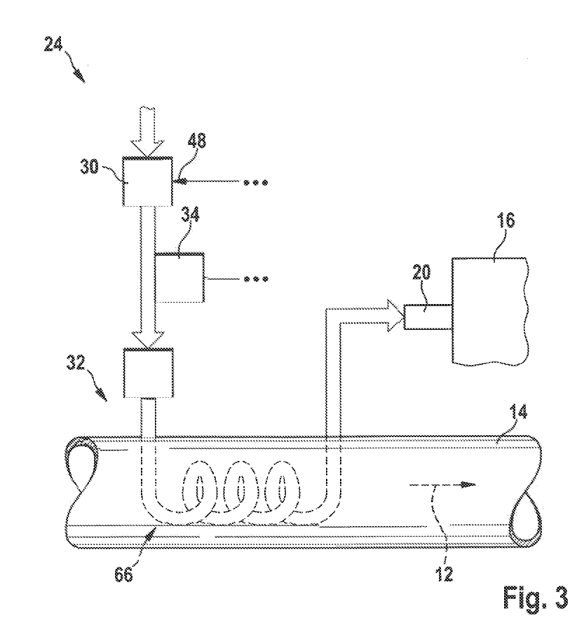

In FIG. 3, an alternative exemplary embodiment of the inventive device 10 is shown, which reflects an advantageous implementation of the air heater 32. The heating of the air is thereby ensured by at least a part 66 of the air heater 32 being disposed within the exhaust gas duct 14. The part 66 of the air heater 32 disposed in the exhaust gas flow 12 is embodied as a heat exchanger and uses the heat energy contained in said exhaust gas flow 12.

1. A device for measuring at least one exhaust gas component in an exhaust gas duct (14) of a combustion process, comprising at least one exhaust gas probe (16), characterized in that the exhaust gas probe (16) has at least one air connection (20), at least one air duct (60) leading past a sensing element (64) of said exhaust gas probe (16) and connected to the air connection (20) as well as at least one air outlet opening (22).

2. The device according to claim 1, characterized in that an air supply device (24) is provided, which is connected to the at least one air connection (20) of the exhaust gas probe (16).

3. The device according to claim 2, characterized in that the air supply device (24) contains a valve (30) to permit or block flow of ambient air (26).

4. The device according to claim 2, characterized in that the air supply device (24) contains a compressor (28).

5. The device according to claim 4, characterized in that the compressor (28) is a part of an exhaust gas turbocharger.

6. The device according to claim 2, characterized in that the air supply device (24) contains an air heater (32).

7. The device according to claim 6, characterized in that at least one part (66) of the air heater (32) is disposed in the exhaust gas duct (14).

8. The device according to claim 1, characterized in that the at least one air outlet opening (22) is disposed in a region (18) of the exhaust gas probe (16) protruding into the exhaust gas duct (14).

9. The device according to claim 1, characterized in that a pressure determination device is provided, which ascertains a pressure difference between the exhaust gas duct (14) and the air supply device (24).

10. The device according to claim 9, characterized in that the pressure determination device contains a first pressure sensor (34) disposed at the air supply device (24) and a second pressure sensor (36) disposed at the exhaust gas duct (14).

11. The device according to claim 1 characterized in that at least one of a lambda, NOx and HC sensor is provided as the exhaust gas probe (16).

12. The device according to claim 1 characterized in that a lambda sensor is provided as the exhaust gas probe (16).

13. The device according to claim 1 characterized in that an NOx sensor is provided as the exhaust gas probe (16).

14. The device according to claim 1 characterized in that an HC sensor is provided as the exhaust gas probe (16).

15. The device according to claim 1 characterized in that a lambda, NOx and HC sensor is provided as the exhaust gas probe (16).

16. The device according to claim 1, characterized in that a specially equipped control unit (38) is provided for operating the device.
